(19) United States
(12) Patent Application Publication
(10) Pub. No.: US 2007/0098658 A1
(43) Pub. Date: May 3, 2007

(54) Cosmetic composition textured with a bis-urea derivative with a liquid fatty phase textured with a bis-urea compound

(75) Inventors: Sandrine Chodorowski-Kimmes, Senlis (FR); Aude Livoreil, Paris (FR); Nawel Baghdadli, Massy (FR); Ivan Rodriguez, Cauffry (FR)

Correspondence Address: OLIFF & BERRIDGE, PLC, P.O. BOX 19928, ALEXANDRIA, VA, 22320, US

(73) Assignee: L'Oreal, Paris (FR)

(21) Appl. No.: 11/585,237

(22) Filed: Oct. 24, 2006

Related U.S. Application Data
(60) Provisional application No. 60/752,373, filed on Dec. 22, 2005.

(30) Foreign Application Priority Data
Oct. 24, 2005 (FR) 05 53231

(51) Int. Cl. A61K 8/00

(52) U.S. Cl. 424/70.1

(57) ABSTRACT

A cosmetic composition textured with a bis-urea derivative with a liquid fatty phase textured with a bis-urea compound includes, in a physiologically acceptable medium, at least one liquid fatty acid phase, textured with at least one compound of general formula (I): 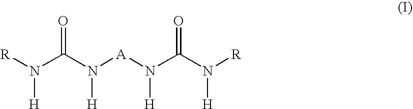 in which A is a phenylene radical.

### CROSS-REFERENCE TO RELATED APPLICATIONS

This non provisional application claims the benefit of French Application No. 05 53231 filed on Oct. 24, 2005 and U.S. Provisional Application No. 60/752,373 filed on Dec. 22, 2005, the entire disclosure of which are incorporated herein by reference.

### BACKGROUND

The present invention relates to cosmetics and more particularly to a cosmetic composition textured with a bis-urea derivative with a liquid fatty phase textured with a bis-urea compound.

To structure oils and give them the desired texture or viscosity, it is known to those skilled in the art to use organogelling agents. Organogelling agents modify the molecular interactions in the oil and change its physical and/or chemical characteristics. However, the dissolution of these organogelling molecules in an oil or an oil mixture often requires a high temperature, which may give rise to additional heating costs and above all, may be incompatible with the presence of heat-sensitive molecules. Furthermore, the gel thus obtained does not have the required stability over time.

The use of certain bis-ureas as organogelling agents has been envisioned in documents WO 02/47628, JP 2003-064346, and JP 10-236981. These documents describe, inter alia, the use of bis-ureas for texturing cosmetic or non-cosmetic media. Many articles also exist describing organic molecules functionalized with one or more ureas, in particular the articles by Bouteiller et al., in New J. Chem., 2000, 24, 845-848; Langmuir, 2002, 18, 7218-7222 and J. Am. Chem. Soc. 2003, 125, 13148-13154 describing the use of certain bis-ureas in organic solvents such as toluene, carbon tetrachloride or dodecane for the purposes of gelling these solvents. Another example that may also be mentioned is Hanabusa K. et al. which describes the behaviour of bis-urea molecules as organogelling agents (Langmuir 2003, 19(21), 8622-8624). As regards the article by Hamilton et al. in Tetrahedron Letters, 1998, 39, 7447-7450, this describes the ability of certain bis-urea derivatives to behave like gelling agents in certain organic solvents.

However, all the bis-ureas described in these documents do not dissolve at room temperature and/or in any oils, especially cosmetic oils.

In point of fact, to identify a gelling agent capable of satisfactorily texturing a particular cosmetic composition and capable of being dissolved at room temperature in the oils of the composition, many tests must generally be performed beforehand.

There is thus a need for organogelling molecules that might be termed universal insofar as they would be effective for texturing, at room temperature, a large number of different cosmetic oils.

### SUMMARY

The object of the present invention is, precisely, to propose novel bis-ureas that meet these requirements.

Consequently, according to one of its aspects, one embodiment relates to a cosmetic composition comprising, in a physiologically acceptable medium, at least one liquid fatty phase containing at least one lipophilic compound selected from the group consisting of

—C₆-C₃₂ monoalcohols,

branched C₆-C₃₂ alkanes,

linear C₁₃-C₄₈ alkanes, and

bifunctional oils, comprising two functions chosen from ester and/or amide, containing from 6 to 30 carbon atoms and 4 heteroatoms selected from the group consisting of O and N,
wherein the fatty phase is textured with an effective amount of at least one compound of general formula (I): 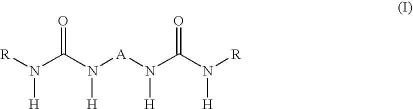
in which:

A is a group of formula 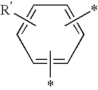
with R′ being a linear or branched C₁ to C₄ alkyl radical and the *s symbolizing the points of attachment of the group A to each of the two nitrogen atoms of the rest of the compound of general formula (I), and

R is a saturated or unsaturated, non-cyclic, mono-branched C₆ to C₁₅ alkyl radical whose hydrocarbon-based chain is optionally interrupted with 1 to 3 heteroatoms selected from the group consisting of O, S and N, or a salt or isomer thereof.

The inventors have discovered, unexpectedly, that the bis-urea compounds as defined above are gelling agents of choice for satisfying the need for universal organogelling molecules stated previously.

According to another of its aspects, another embodiment relates to the use of at least one bis-urea compound of formula (I) as described above for texturing a cosmetic composition.

According to yet another of its aspects, another embodiment relates to a process for making up and/or caring for a keratin material, especially the skin, especially of the body or the face, the eyelashes, the eyebrows, the nails and/or the hair, comprising the application to the surface to be treated of a cosmetic composition comprising at least one bis-urea compound of formula (I) as described above.

### DETAILED DESCRIPTION OF EMBODIMENT

1) Bis-Ureas

As stated above, the bis-urea compounds more particularly considered according to the invention correspond to the general formula (I) below: 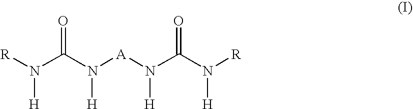
in which:

A is a group of formula: 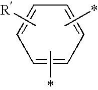
with R′ being a linear or branched C₁ to C₄ alkyl radical and the *s symbolizing the points of attachment of the group A to each of the two nitrogen atoms of the rest of the compound of general formula (I), and

R is a saturated or unsaturated, non-cyclic, mono-branched C₆ to C₁₅ alkyl radical whose hydrocarbon-based chain is optionally interrupted with 1 to 3 heteroatoms selected from the group consisting of O, S and N,

or a salt or isomer thereof.

According to one preferred embodiment, the group represented by A is a group of formula: 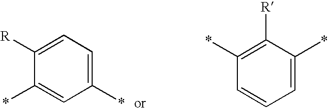
with R′ and the *s being as defined above.

In particular, R′ may be a methyl group, and the group A is then more particularly a group of formula: 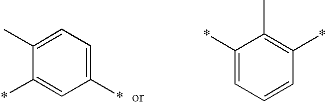
with the *s being as defined above.

According to a first embodiment of the invention, R may be chosen from the mono-branched radicals of general formula CₙH₂ₙ₊₁, n being an integer ranging from 6 to 15, in particular from 7 to 9 or even equal to 8.

Thus, the two groups R of the compound of formula (I) may represent, respectively, a group: 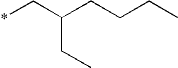
with * symbolizing the point of attachment of each of the groups R to each of the nitrogen atoms of the rest of the compound of general formula (I).

According to a second embodiment of the invention, R may be chosen from the mono-branched radicals of general formula Cₘ₋ₚH₂ₘ₊₁₋₂ₚXₚ, p being equal to 1, 2 or 3, preferably equal to 1; m being an integer ranging from 6 to 15, preferably from 10 to 14, in particular from 10 to 12, or even equal to 11; and X representing sulfur and/or oxygen atoms, in particular oxygen atoms.

More particularly, R may be a radical of formula C_{m′}H_{2m′}X−(C_{p′}H_{2p′}X′)ᵣ—CₓH₂ₓ₊₁, in which X and X′ are, independently of each other, an oxygen or sulfur atom, preferably oxygen; r is 0 or 1; m′, p′ and x are integers such that their sum ranges from 6 to 15, in particular from 10 to 12, or even is equal to 11; and it being understood that at least one of the carbon-based chains C_{m′}H_{2m′}, C_{p′}H_{2p′}, or CₓH₂ₓ₊₁ is branched.

Preferably, it is the chain CₓH₂ₓ₊₁ that is branched, preferably r is equal to 0, preferably m′ is an integer ranging from 1 to 10, especially from 2 to 6, in particular is equal to 3; and/or preferably x is an integer ranging from 4 to 16, especially from 6 to 12 and in particular is equal to 8.

Thus, the two groups R of the compound of formula (I) may represent, respectively, a group: 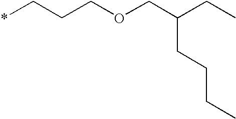
with * symbolizing the point of attachment of each of the groups R to each of the nitrogen atoms of the rest of the compound of general formula (I).

Such compounds may be present in the compositions according to the invention as mixtures with isomers, especially positional isomers on the group A, especially in 95/5 or 80/20 proportions.

As emerges from the examples below, the presence of one or the other of these radicals in the molecule of general formula (I) proves to be particularly advantageous for giving a universal nature to the corresponding bis-urea derivatives.

As non-limiting representations of the compounds that are most particularly suitable for the invention, mention may be made more particularly of the following compounds, used pure or as a mixture: 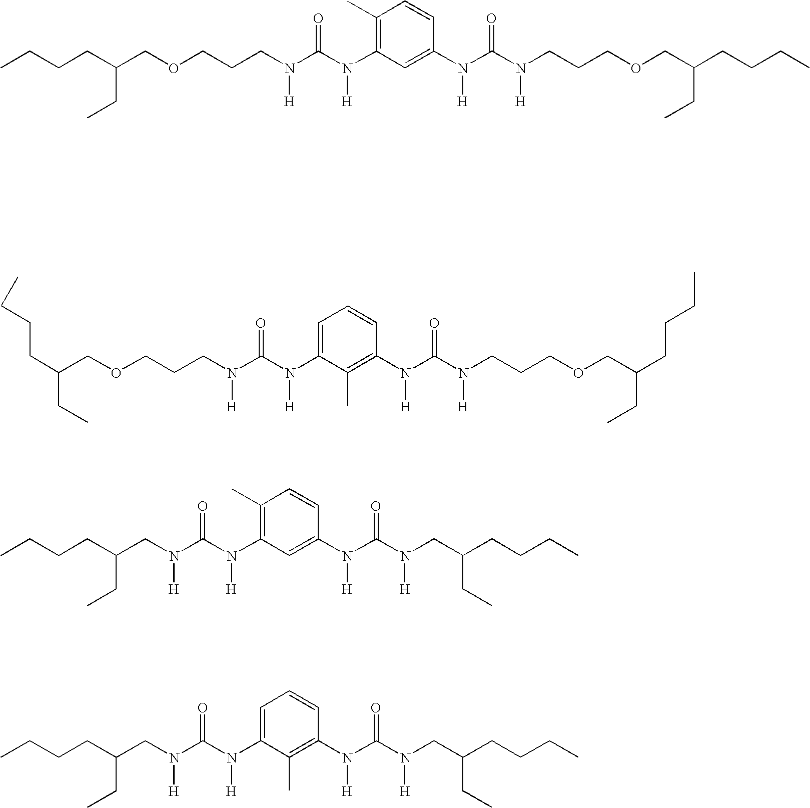
and the salts thereof.

The term “particularly suitable in the context of the embodiments” means that the compound of general formula (I), alone or as a mixture in all proportions, may be dissolved in a wide variety of oils and that it proves to be effective for texturing the oil or oil mixture under consideration and for giving it the desired physical and/or chemical properties.

The term “physiologically acceptable” denotes a medium free of toxicity, which is compatible with application to the skin, the lips and/or the integuments of living beings, and in particular, of human beings. Consequently, the compositions according to the invention are free of compounds that are incompatible with and/or not tolerated for application to the skin, the lips and/or the integuments.

The term “effective amount” denotes the amount that is necessary and sufficient to obtain texturing of the oil or oil mixture under consideration in the composition according to the various embodiments.

The term “textured liquid fatty phase” means that the fatty phase takes the form of a gel or a thickened liquid. It may flow under its own weight. It may be deformed at constant volume if a stress is exerted thereon.

This texturing is reflected in particular by an increase in the viscosity due especially to the introduction of at least one compound of general formula (I).

For example, the compositions may contain from 0.01% to 20% by weight, preferably from 0.1% to 15% by weight or from 1% to 10% by weight and more preferably from 2% to 8% by weight of compound(s) of formula (I) relative to the total weight of the composition.

The effective amount of compound(s) of formula (I) may represent from 0.01% to 20%, preferably from 0.05% to 10%, for example from 0.1% to 5%, or more preferably from 0.05% to 3%, all by weight of the liquid fatty phase.

For obvious reasons, this effective amount is liable to vary significantly depending, firstly, on the nature of the substituent R of the bis-urea derivative, its position, whether or not it is used in pure form or as a mixture with other bis-urea derivatives of formula (I), and, secondly, the nature of the liquid fatty phase.

In general, the compound of general formula (I) according to the invention is derived from the reaction between at least one diisocyanate of formula: 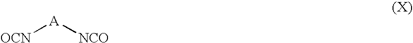
and a primary amine of formula: 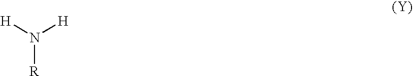
with A and R as defined above.

Where appropriate, the various diisocyanates (X) may be positional isomers of the substituent R′ on the group A, especially in 95/5 or 80/20 proportions.

Preferably, the amine (Y) used is in a mole ratio of 2 to 3 equivalents, preferably 2.1 to 2.5 or more preferably 2.2 equivalents per one equivalent of diisocyanate(s) (X). The general reaction scheme is as follows:

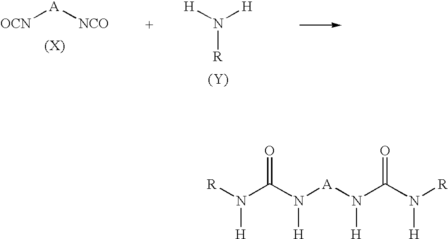

The reaction is generally performed under an inert atmosphere, for example under argon, in anhydrous medium with, for example, a reaction medium temperature that is maintained between 15° C. and 40° C. and preferably between 18° C. and 25° C.

The diisocyanate(s) (X) may be dissolved in an anhydrous solvent such as tetrahydrofuran, 2-methyltetrahydrofuran, N-methylpyrrolidone, butyl acetate or methyl ethyl ketone, at a concentration possibly ranging from 1% to 30% by weight, preferably from 2% to 20% or even from 4% to 10% by weight.

A solution containing the amine (Y) is generally prepared in the same solvent as the diisocyanate(s) (X) at a concentration ranging, for example, from 0.1% to 99.9% by mass. The temperature of the reaction medium should preferentially not exceed 40° C. and the concentration of the amine and the rate of addition of the solution containing the amine (Y) should therefore preferentially be adjusted to this necessity. The reaction medium may be left stirring, for example, for 30 minutes to 12 hours. Monitoring of the reaction progress may be performed by infrared spectrometry (especially by observing the disappearance of the NCO band between 2250 and 2280 cm⁻¹). For example, at the end of the reaction, the reaction medium is poured into a large amount of acidified water (especially at pH 3-4 with HCl). A precipitate is then obtained, which is filtered off, washed, for example several times especially with water, and dried under reduced pressure, especially under vacuum or freeze-dried. The precipitate corresponds to the expected compounds of formula (I), and may be characterized by NMR spectrometry (¹H and/or 1³C) and/or by HPLC and may be used as obtained for the texturing of the oily medium under consideration.

The bis-urea or the bis-urea mixture is advantageously soluble at a temperature of less than or equal to 50° C., or even less than or equal to 30° C., and especially at room temperature, in the liquid fatty phase to be textured.

2) Liquid Fatty Phase

The term “liquid fatty phase” means a fatty phase that is liquid at room temperature (25° C.) and atmospheric pressure (760 mm Hg), composed of one or more lipophilic compounds as defined above, and optionally of one or more fatty substances that are liquid at room temperature, also known as oils, which are mutually compatible and physiologically acceptable.

The liquid fatty phase may represent from 1% to 99.99% by weight of the composition, or from 5% to 95%, preferably from 10% to 90% and more preferably from 20% to 50%, all by weight of the composition.

As stated previously, the cosmetic compositions comprise a fatty phase comprising at least one compound selected from the group consisting of

—C₆-C₃₂ monoalcohols,

branched C₆-C₃₂ alkanes,

linear C₁₃-C₄₈ alkanes, and

bifunctional oils, comprising two functions chosen from ester and/or amide and containing from 6 to 30 carbon atoms and 4 heteroatoms chosen from O and N.

The above compounds may also be referred to as “lipophilic compounds”.

According to one embodiment of the invention, the C₆-C₃₂ monoalcohol is a C₈-C₂₈ and preferably C₁₂-C₂₆ monoalcohol and is more preferably octyldodecanol.

According to another embodiment, the branched C₆-C₃₂ alkane is a branched C₈-C₂₈ and preferably C₁₂-C₂₆ alkane and is more preferably isododecane or Parleam of formula —(CH₂—CH(CH₃)ₙ— with n being an integer ranging, for example, from 4 to 8.

According to another embodiment of the invention, the linear C₁₃-C₄₈ alkane is a linear C₁₈-C₄₀ and better still C₂₀-C₃₂ alkane.

According to yet another embodiment, the bifunctional oil contains from 8 to 28 carbon atoms and preferably from 10 to 24 carbon atoms, the amide and/or ester functions of the oil are in the chain, and more preferably the bifunctional oil may be isopropyl N-lauroylsarcosinate, having the following formula: 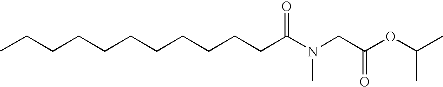
The above oil may be the product sold under the name Eldew SL-205® from Ajinomoto.

The lipophilic compound(s) preferably represent(s) from 20% to 100%, or even from 40% to 99% and more preferably from 60% to 95%, all by weight of the liquid fatty phase.

In addition to the lipophilic compound(s) contained in the liquid fatty phase, this fatty phase may also contain one or a mixture of additional oils selected from the group consisting of ester oils, non-silicone oils and silicone oils.

This (these) additional oil(s) may represent from 0.1% to 80%, for example from 1% to 60% or preferably from 5% to 40%, all by weight of the liquid fatty phase.

a. Additional Ester Oil

According to one variant of the embodiments, the liquid fatty phase may also comprise an oil known as an “ester oil”, which is especially chosen from esters of monocarboxylic acids with monoalcohols and polyalcohols.

Advantageously, the ester corresponds to formula (II) below:
R₁—CO—O—R₂ (II)
in which:

R₁ represents a linear or branched alkyl radical of 1 to 40 carbon atoms and preferably of 7 to 19 carbon atoms, optionally comprising one or more ethylenic double bonds, optionally substituted, and the hydrocarbon-based chain of which may be interrupted with one or more heteroatoms chosen from N and O and/or one or more carbonyl functions, and

R₂ represents a linear or branched alkyl radical of 1 to 40 carbon atoms, preferably of 3 to 30 carbon atoms and more preferably of 3 to 20 carbon atoms, optionally comprising one or more ethylenic double bonds, optionally substituted, and the hydrocarbon-based chain of which may be interrupted with one or more heteroatoms chosen from N and O and/or one or more carbonyl functions.

The term “optionally substituted” means that R₁ and/or R₂ may bear one or more substituents chosen, for example, from groups comprising one or more heteroatoms chosen from O or N, such as amino, amine, alkoxy or hydroxyl.

Examples of groups R₁ are those derived from fatty acids, preferably higher fatty acids, selected from the group consisting of acetic acid, propionic acid, butyric acid, caproic acid, caprylic acid, pelargonic acid, capric acid, undecanoic acid, lauric acid, myristic acid, palmitic acid, stearic acid, isostearic acid, arachidic acid, behenic acid, oleic acid, linolenic acid, linoleic acid, oleostearic acid, arachidonic acid and erucic acid, and mixtures thereof.

Examples of esters that may be used in the fatty phases of the compositions of the invention are C₈-C₄₈ esters, optionally incorporating in their hydrocarbon-based chain one or more heteroatoms from N and O and/or one or more carbonyl functions.

Non-limiting illustrations of these esters include, but are not limited to, purcellin oil (cetostearyl octanoate), isononyl isononanoate, isopropyl myristate, 2-ethylhexyl palmitate, 2-octyldodecyl stearate, 2-octyldodecyl erucate, isostearyl isostearate, and alcohol or polyalcohol heptanoates, octanoates, decanoates or ricinoleates, for example of fatty alcohols.

Preferably, R₁ is an unsubstituted branched alkyl group of 4 to 14 carbon atoms and preferably of 8 to 10 carbon atoms and R₂ is an unsubstituted branched alkyl group of 5 to 15 carbon atoms and preferably of 9 to 11 carbon atoms.

a. Additional Non-Silicone Oil

The liquid fatty phase of the compositions may also contain one or more non-silicone oils different from the lipophilic compounds and from the ester oils described above. These non-silicone oils may be selected from the group consisting of hydrocarbon-based oils and volatile ethers.

The non-silicone oil may also be chosen from fluoro oils such as perfluoropolyethers, perfluoroalkanes, for instance perfluorodecalin, perfluoroadamantanes, perfluoroalkyl phosphate monoesters, diesters and triesters, and fluorinated ester oils.

The liquid fatty phase may also contain other non-silicone oils, for example polar oils such as:

(i) hydrocarbon-based plant oils with a high triglyceride content consisting of fatty acid esters of glycerol in which the fatty acids may have varied chain lengths, these chains possibly being linear or branched, and saturated or unsaturated; these oils include, but are not limited to, wheatgerm oil, corn oil, sunflower oil, shea oil, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grapeseed oil, black currant pip oil, evening primrose oil, millet oil, barley oil, quinoa oil, olive oil, rye oil, safflower oil, candlenut oil, passionflower oil or musk rose oil; or alternatively caprylic/capric acid triglycerides, for instance those sold by the company Stearines Dubois or those sold under the names Miglyol 810, 812 and 818 by the company Dynamit Nobel,

(ii) C₆ to C₄₀ synthetic ethers,

(iii) C₈-C₃₂ fatty acids, for instance oleic acid, linoleic acid or linolenic acid, and

(iv) mixtures thereof.

The liquid fatty phase may also contain polar oils other than the lipophilic compounds mentioned above, such as linear or branched hydrocarbons or fluorocarbons, of synthetic or mineral origin, for instance liquid paraffins (such as C₈-C₁₆ isoparaffins or isohexadecane) and derivatives thereof, petroleum jelly, polydecenes, and hydrogenated polyisobutenes such as squalane, and mixtures thereof.

a. Silicone oil

According to one particular embodiment, the liquid fatty phase may also comprise one or more silicone oil(s).

These oils may be volatile or non-volatile. For the purposes of the invention, the volatile oils have at room temperature (25° C.) and atmospheric pressure (760 mm Hg) a vapour pressure ranging from 0.02 mm Hg to 300 mm Hg (2.66 Pa to 40 000 Pa) and preferably ranging from 0.1 mm Hg to 90 mm Hg (13 Pa to 12 000 Pa). The non-volatile oils then correspond to a vapour pressure of less than 0.02 mm Hg (2.66 Pa).

The silicone oil may be chosen from linear or cyclic volatile silicone oils, such as linear or cyclic polydimethylsiloxanes (PDMS) containing from 3 to 7 silicon atoms.

Examples of such volatile oils include, but are not limited to, compounds such as octyl trimethicone, hexyl trimethicone, decamethylcyclopentasiloxane (or D5), octamethylcyclotetrasiloxane, cyclotetradimethylsiloxane (or D4), dodecamethylcyclohexasiloxane (or D6), decamethyltetrasiloxane (or L4) KF 96 A from Shin Etsu, PDMS (polydimethylsiloxane) DC 200 (1.5 cSt) from Dow Corning, PDMS DC 200 (2 cSt) from Dow Corning, PDMS DC 200 (5 cSt) from Dow Corning, and PDMS DC 200 (3 cSt) from Dow Corning, and/or mixtures thereof.

Mention may also be made of heptamethyloctyltrisiloxane, dodecamethylpentasiloxane and polymethylcetyldimethylsiloxane, and/or mixtures thereof.

The volatile silicone oil may also be chosen from the group of fluorosilicone oils such as silicones containing alkyl and perfluoroalkyl groups, silicones containing oxyethylene/oxypropylene (OE/PP) side groups and perfluoro groups, silicones containing perfluoro or polyfluoro side groups and glycerolated side groups, and perfluoroalkylmethylphenylsiloxanes.

The non-volatile silicone oils may be polydimethylsiloxanes, polyalkylmethylsiloxanes, dimethicone copolyols, alkylmethicone copolyols, cetyl dimethicone, silicones containing alkylglyceryl ether groups, silicones containing amine side groups and dilauroyltrimethylol propane siloxysilicate. The alkyl groups of these oils especially contain from 2 to 24 carbon atoms.

The non-volatile silicone oils that may be used include, but are not limited to, linear non-volatile polydimethylsiloxanes (PDMS) that are liquid at room temperature; polydimethylsiloxanes comprising alkyl, alkoxy or phenyl groups, which are pendent and/or at the end of a silicone chain, these groups each containing from 2 to 24 carbon atoms; phenyl silicones, for instance phenyl trimethicones, phenyl dimethicones, phenyl trimethylsiloxy diphenylsiloxanes, diphenyl dimethicones, diphenyl methyldiphenyl trisiloxanes, 2-phenyl ethyl trimethylsiloxysilcates, fluorosilicones with groups that are pendent or at the end of a chain, containing from 1 to 12 carbon atoms, all or some of the hydrogen atoms of which are substituted with fluorine atoms, dimethiconols, and/or mixtures thereof.

The inventors have found that combinations of at least one bis-urea of formula (I) in which the two groups R are represented, respectively, by a group: 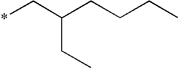
with * symbolizing the point of attachment of each of the groups R to each of the nitrogen atoms of the rest of the compound of general formula (I), with at least one lipophilic compound as defined above, and especially a fatty phase containing isododecane, octyldodecanol, isopropyl N-lauroylsarcosinate, Parleam and/or a mixture thereof, give particularly satisfactory results.

Similarly, combinations of at least one bis-urea of formula (I) in which the two groups R are represented, respectively, by a group: 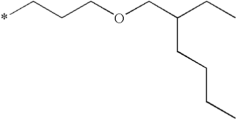
with * symbolizing the point of attachment of each of the groups R to each of the nitrogen atoms of the rest of the compound of general formula (I), with at least one lipophilic compound as defined above, and especially a fatty phase containing isododecane, octyldodecanol, Parleam, isopropyl N-lauroylsarcosinate and/or a mixture thereof, also give particularly satisfactory results.

3) Other Fatty Substances

The compositions may also comprise at least one solid fatty substance, which may be chosen from waxes and/or pasty compounds.

More particularly, the compositions may include from 0.1% to 40% by weight, preferably from 0.1% to 30% by weight and more preferably from 0.5% to 25% by weight of solid fatty substance(s) relative to the total weight of the composition.

The term “synthetic lipophilic compound” means a compound that is solid at room temperature (25° C.), with a reversible solid/liquid change of state, having a melting point of greater than or equal to 30° C. and better still greater than 45° C., which may be up to 120° C.

As waxes that may be used in the first embodiment, mention may be made of beeswax, carnauba wax, candelilla wax, paraffin, microcrystalline waxes, ceresin or ozokerite; synthetic waxes, for instance polyethylene wax or Fischer Tropsch wax, and silicone waxes, for instance alkyl or alkoxydimethicones containing from 16 to 45 carbon atoms.

The compositions may also contain a micronized wax, also known as a micro wax.

As a guide, the compositions according to the invention may contain from 0.1% to 50% by weight and preferably from 1% to 30% by weight of wax relative to their total weight.

4) Aqueous Phase

The composition may, where appropriate, comprise at least one aqueous phase, which may or may not consist essentially of water.

The composition may also comprise a mixture of water and of water-miscible organic solvent (miscibility in water of greater than 50% by weight at 25° C.), for instance lower monoalcohols containing from 1 to 5 carbon atoms such as ethanol or isopropanol, glycols containing from 2 to 8 carbon atoms such as propylene glycol, ethylene glycol, 1,3-butylene glycol or dipropylene glycol, C₃-C₄ ketones and C₂-C₄ aldehydes.

The aqueous phase (water and possibly the water-miscible organic solvent) may be present in an amount ranging from 1% to 95% by weight, preferably from 3% to 80% by weight, and more preferably ranging from 5% to 60% by weight relative to the total weight of the composition.

According to another embodiment, the preparation is free of water.

5) Dyestuffs

According to one embodiment, the composition(s) may also contain at least one organic or inorganic dyestuff, especially of pigment or nacre type.

According to another embodiment, the composition may also contain at least one dyestuff selected from the group consisting of lipophilic dyes, hydrophilic dyes, pigments, nacres and materials with a specific optical effect, and mixtures thereof.

The term “nacres” should be understood as meaning coloured particles of any form, which may or may not be iridescent, especially produced by certain mollusks in their shell or else synthesized, and which have a colour effect by optical interference.

This dyestuff may be present in an amount ranging from 0.01% to 50% by weight relative to the total weight of the composition, preferably from 0.5% to 40%, more particularly from 5% to 25%, more preferably from 0.01% to 20%, in particular from 0.1% to 10% or from 2% to 5% all by weight relative to the total weight of the composition.

6) Other Additives

The composition(s) may also comprise any ingredient usually used in the field under consideration.

Needless to say, a person skilled in the art will take care to select the optional additional ingredients and/or the amount thereof such that the advantageous properties of the composition(s) are not, or are not substantially, adversely affected by the envisioned addition.

Mention may be made especially of fillers, vitamins, thickeners other than those corresponding to the general formula (I), gelling agents other than those corresponding to formula (I), trace elements, softeners, moisturizers, sequestrants, fragrances, acidifying or basifying agents, preserving agents, sunscreens, surfactants, antioxidants, hair loss counteractants, antidandruff agents, propellants, ceramides, cosmetic active agents in general (anti-ageing, canities, hair-loss-counteracting active agents, facial and/or body care active agents, slimming agents, etc.), oxidizing agents, reducing agents (for dyeing) and dermatological active agents, and/or mixtures thereof.

The composition(s) may preferably be in the form of a suspension, a dispersion, a solution, especially an organic solution, a gel, an emulsion, especially an oil-in-water (O/W) or water-in-oil (W/O) or multiple (W/O/W or polyol/O/W or O/W/O) emulsion, or in the form of cream, paste, mousse, vesicular dispersion especially of ionic or nonionic lipids, two-phase or multiphase lotion, spray, powder, stick or wand.

A person skilled in the art may select the appropriate galenical form, and also the method for preparing it, on the basis of his and her general knowledge, taking into account firstly the nature of the constituents used, especially their solubility in the support, and secondly the intended use of the composition.

The composition(s) may be a makeup composition, especially a complexion product such as a foundation, a makeup rouge or an eyeshadow; a lip product such as a lipstick or a lipcare product; a concealer product; a blusher, a mascara or an eyeliner; an eyebrow makeup product, a lip pencil or an eye pencil; a nail product such as a nail varnish or a nailcare product; a body makeup product; a hair makeup product (hair mascara or hair lacquer).

The composition(s) may be used for protecting or caring for the skin of the face, the neck, the hands or the body, especially an anti-wrinkle, anti-fatigue or anti-ageing composition, for making the skin radiant, a moisturizing or treating composition; an antisun or after-sun or artificial tanning composition. The composition(s) may also be a hair product, especially for the dyeing, care or hygiene of the hair, for holding the hairstyle or for shaping the hair. The hair compositions are preferably shampoos, gels, hairsetting lotions, blow-drying lotions, haircare or hair hygiene compositions, or fixing and styling compositions such as lacquers or sprays. The hair products may be packaged in various forms (gels, pastes, creams, etc.). They may also be lotions, especially in vaporizers or pump-dispenser bottles or in aerosol containers in order to apply the composition in vaporized form or in the form of a mousse.

The composition(s) may be manufactured via the known processes generally used in cosmetics or dermatology.

Another embodiment is also a cosmetic treatment process, especially for making up, cleansing, sun-protecting, shaping, dyeing or caring for keratin materials, found in body or facial skin, the nails, the eyebrows, the hair and/or the eyelashes, comprising the application to the keratin materials of a cosmetic composition as defined above.

The examples given below are provided as non-limiting illustrations of the field of the invention.

Materials and Method

Examples 1 to 3 given below differ in the chemical nature of the amine (Y) used in the reaction mixture and also in the starting diisocyanate (X). The symbols (X) and (Y) indicate the reagents in the general reaction scheme presented previously.

Unless otherwise mentioned, the compounds of general formula (I) considered in each of Examples 1 to 3 were prepared according to the same procedure as described below. The specific amounts retained for compounds X and Y are specified in the examples below.

A mixture of tolylene diisocyanate as a solution in THF with 2.2 equivalents of amine is prepared by reaction under argon. The reaction is performed under an inert atmosphere in anhydrous medium with a reaction medium temperature maintained between 15° C. and 40° C.

In parallel, a solution of amine (Y) in THF is prepared. Since the temperature of the reaction medium should not exceed 40° C., the concentration of the amine and the rate of addition of the amine (Y) solution are adjusted to this need. The reaction medium is left stirring while monitoring the reaction progress by infrared spectrometry (disappearance of the NCO band between 2250 and 2280 cm⁻¹).

Once the diisocyanate has completely reacted, water acidified to pH 3 with hydrochloric acid is added to the reaction mixture, and the precipitate obtained is filtered off, washed several times with water and finally dried. A white powder is obtained and is used as obtained, after analysis (for example, HPLC coupled to mass spectrometry).

### EXAMPLE 1

(X)=50 g of tolylene diisocyanate as a mixture of 2,4 isomer and 2,6 isomer in 95/5 proportions,

(Y)=79.6 g of 2-ethylhexylamine:

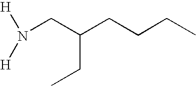

Compound (I) is obtained in the form of a mixture of compounds having the following formulae:

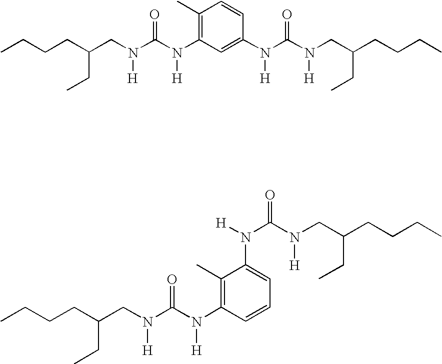

The mole ratio of the isomers is determined by 1H NMR spectrometry and/or by HPLC. The NMR spectra are in accordance with the expected structures. The mixture of product obtained is in the form of a white powder.

### EXAMPLE 2

(X)=19.8 g of tolylene diisocyanate as a mixture of 2,4 isomer and 2,6 isomer in 95/5 proportions.

(Y)=47.2 g of 3-(2-ethylhexyloxy)propylamine:

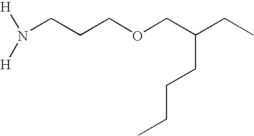

Compound (I) is obtained in the form of a mixture of the following formulae:

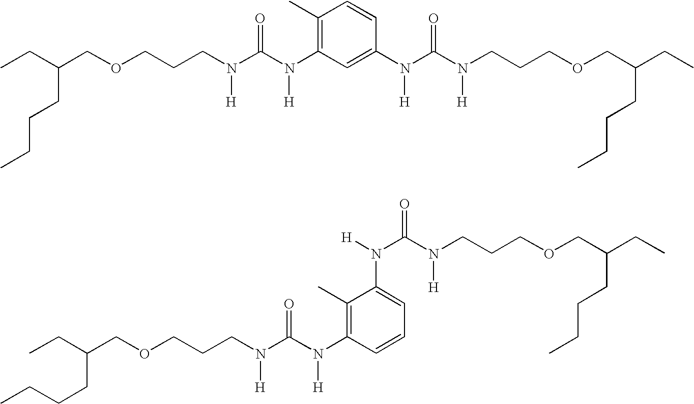

The mole ratio of the isomers is determined by 1H NMR spectrometry and/or by HPLC. The NMR spectra are in accordance with the expected structures. The mixture of product obtained is in the form of a white powder.

### EXAMPLE 3

(X)=8.7 g of tolylene diisocyanate as a mixture of 2,4 isomer and 2,6 isomer in 80/20 proportions.

(Y)=14.2 g of 2-ethylhexylamine:

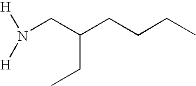

Compound (I) is obtained in the form of a mixture of the following formulae:

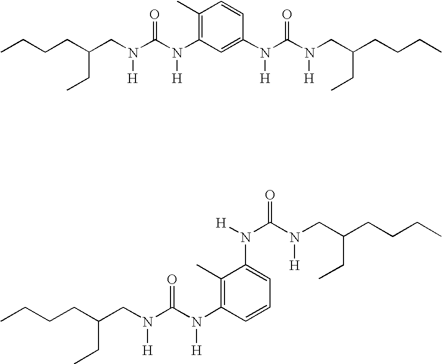

The mole ratio of the isomers is determined by 1H NMR spectrometry and/or by HPLC. The NMR spectra are in accordance with the expected structures. The mixture of products obtained is in the form of a white powder.

### EXAMPLE 4

The mixtures of bis-ureas below were prepared for comparative purposes:

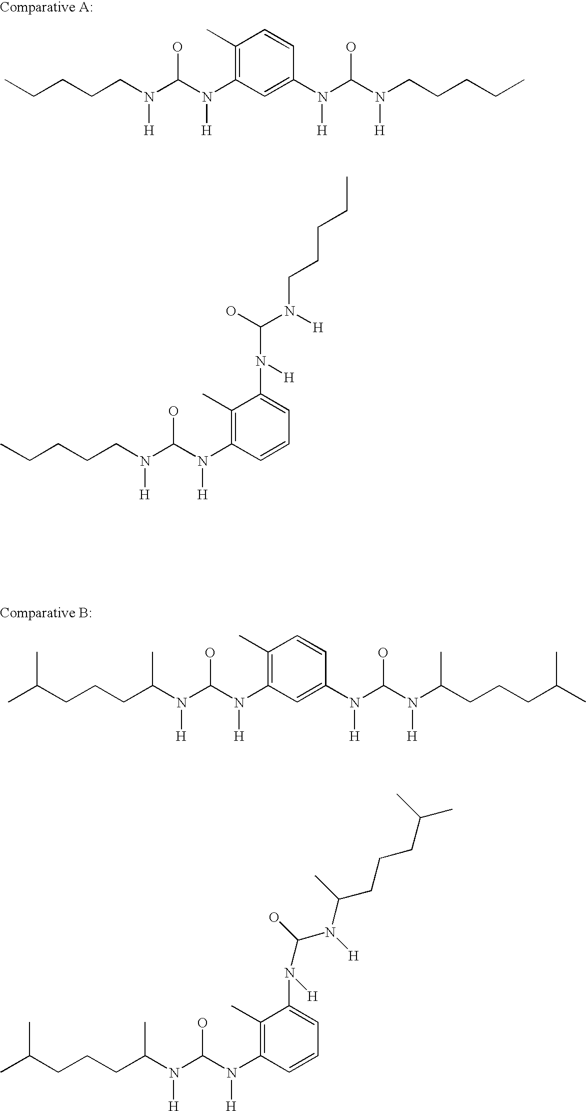

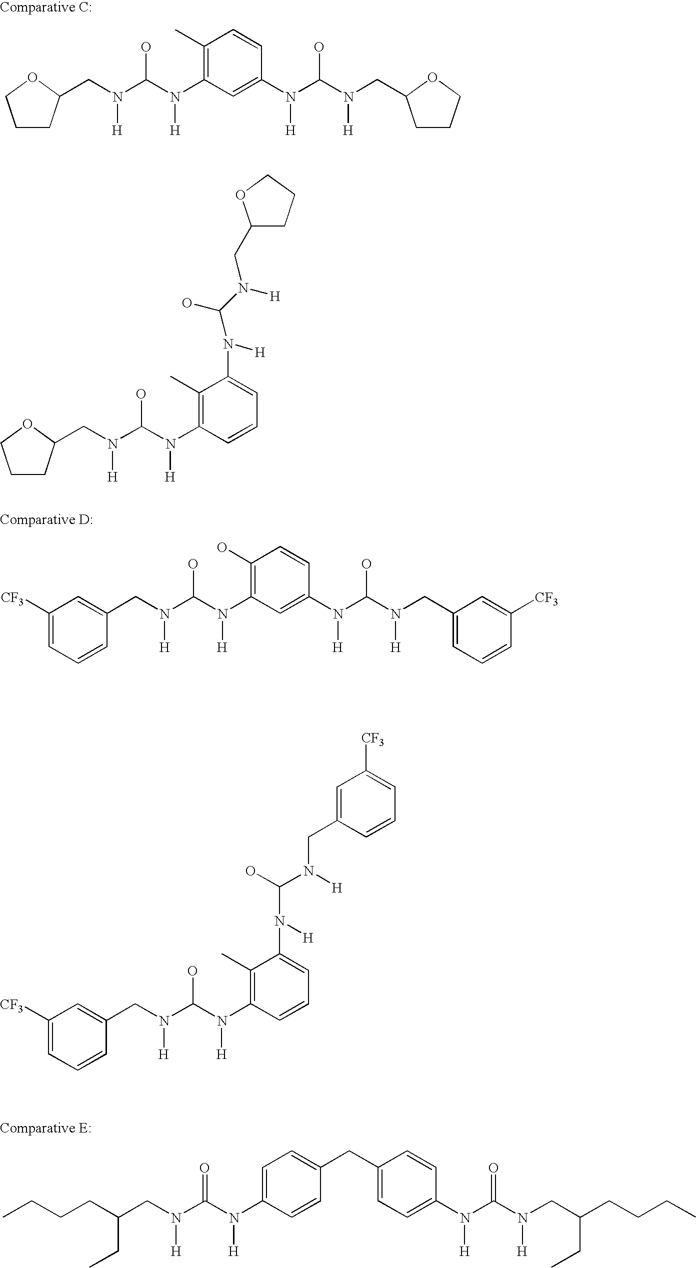

### EXAMPLE 5

The products or product mixtures obtained in each of Examples 1, 2 and 3 and the comparative compounds A, B, C, D and E are tested at a rate of 1% to 2% by weight in 100 ml of the lipophilic compounds under consideration, for their texturing properties at room temperature with respect to a list of lipophilic compounds (isododecane, octyldodecanol, Parleam and isopropyl N-lauroylsarcosinate).

The texturing properties are judged to be satisfactory if the compound or compound mixture dissolves at room temperature in the lipophilic compound and, where appropriate, if it increases the viscosity of the lipophilic compound.

It is noted that only the mixtures of Examples 1, 2 and 3 comprising a compound in accordance with the embodiments prove to be soluble at room temperature at concentrations of up to 10% by weight in isododecane, octyldodecanol and Parleam, and increase the viscosity of these lipophilic compounds.

On the other hand, the derivatives of the comparative examples A, B, C, D and E are not found to be soluble at 1% by weight in these lipophilic compounds, even at 80° C.

Similarly, it is observed that the mixture of Example 1 or 3 is soluble at room temperature at concentrations ranging from 0.5% to 10% in isopropyl N-lauroylsarcosinate and increases the viscosity of this compound.

On the other hand, the derivatives of the comparative examples A and B are not soluble at 1% by weight in this lipophilic compound, even at 80° C., and therefore is not suitable as a structuring agent in this compound.

### EXAMPLE 6

By way of example of oil mixture, compound (I) obtained in Example 1 is dissolved at room temperature at 2% by weight in isododecane (0.5 g of the mixture in 25 g of isododecane). Once a homogeneous gel is obtained, 25 g of silicone oil D5 is added. A gel that is stable over time in an isododecane/D5 mixture is finally obtained.

### EXAMPLE 7

By way of example of oil mixture, compound (I) obtained in Example 3 is dissolved at room temperature at 2% by weight in isododecane (0.5 g of the mixture in 25 g of isododecane). When a homogeneous gel is obtained, 25 g of silicone oil D5 are added thereto. A gel that is stable over time in an isododecane/D5 mixture is finally obtained.

### EXAMPLE 8

### Mascara

The following composition is prepared:

| | Concentration |
|---|---|
| Constituents | (weight %) |
| Beeswax | 9.9 |
| Carnauba wax | 4.52 |
| Paraffin | 2.18 |
| Mexomer PQ 1 | 0 |
| Mexomer PP 2 | 0.75 |
| Compound of Example 1 | 5 |
| Black iron oxide | 2.5 |
| Ultramarine blue | 2.1 |
| Preserving agent | 0.2 |
| Isododecane | qs |

1 Allyl stearate/VA Copolymer
2 Polyvinyl laurate

A gel with entirely satisfactory cosmetic qualities is obtained. A mascara having the required cosmetic qualities is obtained.

What is claimed is:

1. A cosmetic composition comprising, in a physiologically acceptable medium, at least one liquid fatty phase containing at least one lipophilic compound selected from the group consisting of
—C₆-C₃₂ monoalcohols,
branched C₆-C₃₂ alkanes,
linear C₁₃-C₄₈ alkanes, and
bifunctional oils, comprising two functions chosen from ester and/or amide, containing from 6 to 30 carbon atoms and 4 heteroatoms selected from the group consisting of O and N,
wherein the fatty phase is textured with an effective amount of at least one compound of general formula (I): 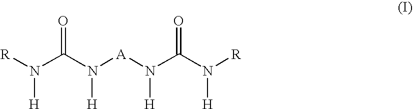
in which:
A is a group of formula: 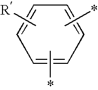
R′ is a linear or branched C₁ to C₄ alkyl radical and the *s symbolize the points of attachment of the group A to each of the two nitrogen atoms of the rest of the compound of general formula (I), and
R is a saturated or unsaturated, non-cyclic, mono-branched C₆ to C₁₅ alkyl radical,
or a salt or isomer thereof.

2. The composition according to claim 1, wherein the hydrocarbon-based chain of R is interrupted with 1 to 3 heteroatoms selected from the group consisting of O, S and N.

3. The composition according to claim 1, wherein the group represented by A is a group of formula:

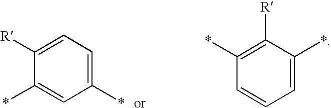

4. The composition according to claim 3, wherein the group A is a group of formula:

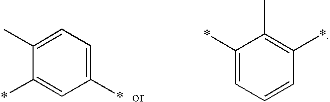

5. The composition according to claim 1, wherein the group R is of general formula CₙH₂ₙ₊₁ with n being an integer ranging from 6 to 15.

6. The composition according to claim 5, wherein n ranges from 7 to 9.

7. The composition according to claim 6, wherein the two groups R represent, respectively; a group: 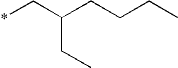
with * symbolizing the point of attachment of each of the groups R to each of the nitrogen atoms of the rest of the compound of general formula (I).

8. The composition according to claim 1, wherein the group R is of general formula Cₘ₋ₚH₂ₘ₊₁₋₂ₚXₚ, p is equal to 1, 2 or 3, m is an integer ranging from 6 to 15, and X is a sulfur and/or oxygen atom.

9. The composition according to claim 8, wherein the group R is of formula C_{m′}H_{2m′}X—(C_{p′}H_{2p′}X′)ᵣ—CₓH₂ₓ₊₁, in which X and X′ are, independently of each other, an oxygen or sulfur atom, r is 0 or 1 and m′, p′ and x are integers such that their sum ranges from 6 to 15.

10. The composition according to claim 9, wherein r is equal to 0.

11. The composition according to claim 9, wherein the chain CₓH₂ₓ₊₁ is branched.

12. The composition according to claim 9, wherein m′ is an integer ranging from 1 to 10.

13. The composition according to claim 9, wherein x is an integer ranging from 4 to 16.

14. The composition according to claim 9, wherein m′ or the sum of m′, p′ and x ranges from 10 to 12.

15. The composition according to claim 14, wherein the two groups R represent, respectively, a group: 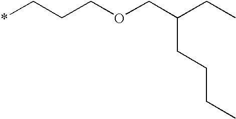
with * symbolizing the point of attachment of each of the groups R to each of the nitrogen atoms of the rest of the compound of general formula (I).

16. The composition according to claim 1, comprising at least one compound selected from the group consisting of 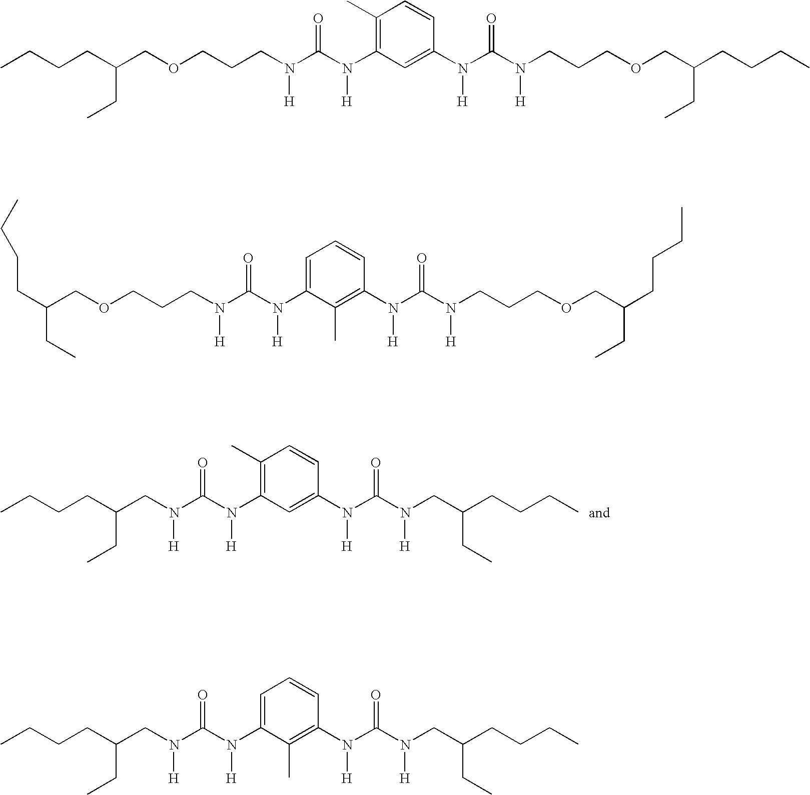
salts thereof and isomers thereof.

17. The composition according to claim 1, comprising about 2% to about 8% by weight of compound(s) of general formula (I) relative to the total weight of the composition.

18. The composition according to claim 1, wherein the compound(s) of general formula (I) represent(s) less than about 3% by weight of the liquid fatty phase.

19. The composition according to claim 1, wherein the C₆-C₃₂ monoalcohol is a C₈-C₂₈ monoalcohol.

20. The composition according to claim 19, wherein the C₆-C₃₂ monoalcohol is octyldodecanol.

21. The composition according to claim 1, wherein the branched C₆-C₃₂ alkane is a branched C₈-C₂₈ alkane.

22. The composition according to claim 21, wherein the C₆-C₃₂ alkane is isododecane or Parleam of formula —(CH₂—CH(CH₃)ₙ— with n being an integer ranging from 4 to 8.

23. The composition according to claim 1, wherein the linear C₁₃-C₄₈ alkane is a linear C₁₈-C₄₀ alkane.

24. The composition according to claim 1, wherein the bifunctional oil contains from 8 to 28 carbon atoms.

25. The composition according to claim 1, wherein the amide and ester functions of the bifunctional oil are in the chain.

26. The composition according to claim 25, wherein the bifunctional oil is isopropyl N-lauroylsarcosinate.

27. The composition according to claim 1, wherein the lipophilic compound(s) represent(s) from about 20% to about 100% by weight of the liquid fatty phase.

28. The composition according to claim 27, wherein the lipophilic compounds(s) represent(s) from about 60% to about 95% by weight of the liquid fatty phase.

29. The composition according to claim 1, wherein the liquid fatty phase comprises at least one ester oil having the formula (II)
R₁—CO—O—R₂ (II)
in which:
R₁ represents a linear or branched alkyl radical of 1 to 40 carbon atoms, and
R₂ represents a linear or branched alkyl radical of 1 to 40 carbon atoms.

30. The composition according to claim 1, wherein the liquid fatty phase comprises at least one hydrocarbon-based oil or one volatile ether.

31. The composition according to claim 1, wherein the liquid fatty phase comprises a silicone oil.

32. The composition according to claim 1, wherein the liquid fatty phase contains at least isododecane, octyldodecanol, isopropyl N-lauroylsarcosinate, Parleam and/or a mixture thereof and at least one bis-urea of formula (I) in which the two groups R represent, respectively, a group: 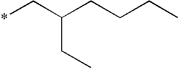
with * symbolizing the point of attachment of each of the groups R to each of the nitrogen atoms of the rest of the compound of general formula (I).

33. The composition according to claim 1, wherein the liquid fatty phase contains at least isododecane, octyldodecanol, Parleam, isopropyl N-lauroylsarcosinate and/or a mixture thereof and at least one bis-urea of formula (I) in which the two groups R represent two groups: 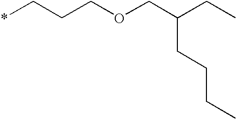
with * symbolizing the point of attachment of each of the groups R to each of the nitrogen atoms of the rest of the compound of general formula (I).

34. The composition according to claim 1, wherein the compound of general formula (I) is derived from the reaction between at least one diisocyanate of formula: 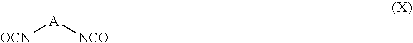
and an amine of formula: 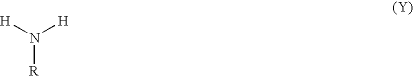
with A and R as defined in claim 1.

35. The composition according to claim 1, also comprising a dyestuff.

36. The composition according to claim 1, also comprising a filler, a pigment or mixtures thereof.

37. The composition according to claim 1, also comprising at least one cosmetic or dermatological active agent.

38. The composition according to claim 1, also comprising at least one additive selected from the group consisting of water, trace elements, softeners, moisturizers, sequestrants, fragrances, acidifying agents, basifying agents, preserving agents, sunscreens, surfactants, antioxidants, hair loss counteractants, antidandruff agents, propellants, ceramides, and mixtures thereof.

39. A method for texturing a cosmetic composition comprising:
(a) providing, in a physiologically acceptable medium, at least one liquid fatty phase containing at least one lipophilic compound selected from the group consisting of
—C₆-C₃₂ monoalcohols,
branched C₆-C₃₂ alkanes,
linear C₁₃-C₄₈ alkanes, and
bifunctional oils, comprising two functions chosen from ester and/or amide, containing from 6 to 30 carbon atoms and 4 heteroatoms selected from the group consisting of O and N, and
(b) texturizing the fatty phase with an effective amount of at least one compound of general formula (I): 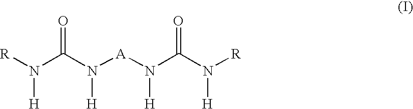
in which:
A is a group of formula: 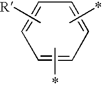
R′ is a linear or branched C₁ to C₄ alkyl radical and the *s symbolize the points of attachment of the group A to each of the two nitrogen atoms of the rest of the compound of general formula (I), and
R is a saturated or unsaturated, non-cyclic, mono-branched C₆ to C₁₅ alkyl radical

40. A process for caring for and/or making up a keratin material in a mammal, comprising the application to a surface of the mammal to be treated of a composition according to claim 1.

41. The process of claim 40 wherein the keratin material is found in the skin.
